(19) United States
(12) Patent Application Publication
(10) Pub. No.: US 2009/0054975 A1
(43) Pub. Date: Feb. 26, 2009

(54) DEPLOYMENT DEVICE FOR CARDIAC SURGERY

(75) Inventors: Pedro del Nido, Boston, MA (US); Yoshihiro Suematsu, Boston, MA (US); Jeremy Cannon, Boston, MA (US); Franz Freudenthal, La Paz (BO)

Correspondence Address: Kenneth I. Kohn;KOHN & ASSOCIATES, 30500 Northwestern Highway, Suite 410, Farmington, MI, 48334, US

(73) Assignee: CHILDREN'S MEDICAL CENTER CORPORATION, Boston, MA (US)

(21) Appl. No.: 10/588,584

(22) PCT Filed: Feb. 7, 2005
(86) PCT No.: PCT/US2005/003739

§ 371 Date: Nov. 4, 2008

Related U.S. Application Data
(60) Provisional application No. 60/542,335, filed on Feb. 6, 2004.

(51) Int. Cl. A61F 2/24; A61F 2/02

(52) U.S. Cl. 623/2.11; 623/23.72

(57) ABSTRACT

A deployment device for deploying a material into a patient, said deployment device having a housing and a placement device including a retracted condition within the housing for holding a material, in a collapsed condition, within the housing and an extended condition from the housing for disposing and releasing the material at a predetermined site in an uncollapsed condition. A method of deploying a material by placing the placement device in an extended condition and affixing the material to the extended placement device, retracting the placement device into the housing with the material in a collapsed condition, extending the placement device, and placing the material at a predetermined site in an uncollapsed condition.

### BACKGROUND OF THE INVENTION

1. Technical Field

Generally, the present invention relates to a device for use in cardiac surgery. More specifically, the present invention relates to a deployment device for use in cardiac surgery.

2. Description of the Related Art

Minimally invasive surgery has enabled physicians to carry out numerous surgical procedures with less pain and disability than conventional, open surgery. In performing minimally invasive surgery, the surgeon makes a number of small incisions through the body wall to obtain access to the tissues requiring treatment. Typically, a trocar, which is a pointed, piercing device, is delivered into the body with a cannula. After the trocar pierces the abdominal or thoracic wall, it is removed and the cannula is left with one end in the body cavity, where the operation is to take place, and the other end opening to the outside. The cannula typically has a small inside diameter, generally 3-10 millimeters. A number of such cannulas can be inserted for any given operation.

A viewing instrument, typically including a miniaturized video camera, is inserted through one of these cannulas and a variety of surgical instruments and retractors are inserted through additional cannulas. The image provided by the viewing device may be displayed on a video screen or television monitor, affording the surgeon enhanced visual control over the instruments. Because a commonly used viewing instrument is called an “endoscope,” this type of surgery is often referred to as “endoscopic surgery.” In the abdomen, endoscopic procedures are commonly referred to as laparoscopic surgery, and in the chest, as thoracoscopic surgery. Abdominal procedures may take place either inside the abdominal cavity (in the intraperitoneal space) or in a space created behind the abdominal cavity (in the retroperitoneal space). The retroperitoneal space is particularly useful for operations on the aorta and spine.

Minimally invasive surgery has virtually replaced open surgical techniques for operations such as cholecystectomy and anti-reflux surgery of the esophagus and stomach. Such minimally invasive surgeries have not occurred in either peripheral vascular surgery or cardiovascular surgery. An important type of vascular surgery includes replacing or bypassing a diseased, occluded, or injured artery. Arterial replacement or bypass grafting has been performed for many years using open surgical techniques and a variety of prosthetic grafts. These grafts are manufactured as fabrics (often from Dacron or Teflon) or are prepared as autografts (from the patient's own tissues) or heterografts (from the tissues of animals). A graft can be joined to the involved artery in a number of different positions, including end-to-end, end-to-side, and side-to-side. This attachment between artery and graft is known as an anastomosis. Constructing an arterial anastomosis is technically challenging for a surgeon in open surgical procedures, and is almost a technical impossibility using minimally invasive techniques.

Minimally invasive surgery is of interest in cardiovascular surgery because of the nature of the tissue of the heart. Cells known as myocytes beat together in unison in a healthy heart when ion channels open and close in an organized manner. Ions pass in and out of the channels, and the change in concentration of ions from within a cell to outside of a cell results in an electrical potential, causing the cell itself to depolarize and repolarize. The depolarization of one cell triggers the cell next to it to depolarize, and thus a cascade effect of depolarization of all the myocytes is triggered and the heart beats. Making several incisions in cardiac tissue can interrupt this cascade during surgery and change the beating of the heart. Keeping incisions to a minimum with minimally invasive techniques enables beating heart surgery to be successful while maintaining the electrical integrity of the heart.

Many factors contribute to the difficulty of performing arterial replacement or bypass grafting. See generally, Wylie, Edwin J. et al., Manual of Vascular Surgery, (Springer-Verlag New York), 1980. One such factor is that the tissues to be joined must be precisely aligned with respect to each other to ensure the integrity and patency of the anastomosis. If one of the tissues is affixed too close to its edge, the suture can rip through the tissue and impair both the tissue and the anastomosis. Another factor is that, even after the tissues are properly aligned, it is difficult and time consuming to pass the needle through the tissues, form the knot in the suture material, and ensure that the suture material does not become tangled. These difficulties are exacerbated by the small size of the artery and graft. The arteries subject to peripheral vascular and cardiovascular surgery typically range in diameter from several millimeters to several centimeters. A graft is typically about the same size as the artery to which it is being attached, thus further complicating the procedure. Another factor contributing to the difficulty of such procedures is the limited time available to complete the procedure. The time to complete an arterial replacement or bypass graft is limited because there is no blood flowing through the artery while the procedure is being done. If blood flow is not promptly restored, sometimes in as little as thirty minutes, the tissue that the artery supplies blood to may experience significant damage, or even death (tissue necrosis). In addition, arterial replacement or bypass grafting is made more difficult by the need to accurately place and space the sutures to achieve a permanent hemostatic seal. Precise placement and spacing of sutures is also required to achieve an anastomosis with long-term patency.

Highly trained and experienced surgeons are able to perform arterial replacement and bypass grafting in open surgery using conventional sutures and suturing techniques. A suture includes a suture needle that is attached or “swedged on” to a long, trailing suture material. The needle must be precisely controlled and accurately placed through both graft and artery. The trailing suture material must be held with proper tension to keep the graft and artery together, and must be carefully manipulated to prevent the suture material from tangling. In open surgery, these maneuvers can usually be accomplished within the necessary time frame, thus avoiding the subsequent tissue damage (or tissue death) that can result from prolonged occlusion of arterial blood flow.

The difficulty of suturing a graft to an artery using minimally invasive surgical techniques has effectively prevented the safe use of this technology in both peripheral vascular and cardiovascular surgical procedures. In some minimally invasive procedures, such as those in the abdominal cavity, the retroperitoneal space, or chest, the space in which the operation is performed is more limited. The exposure to the involved organs is also more, restricted than with open surgery. Moreover, in a minimally invasive procedure, the instruments used to assist with the operation are passed into the surgical field through cannulas. When manipulating instruments through cannulas, it is extremely difficult to position tissues in their proper alignment with respect to each other, pass a needle through the tissues, form a knot in the suture material once the tissues are aligned, and prevent the suture material from becoming tangled. Therefore, although there have been isolated reports of vascular anastomoses being formed by minimally invasive surgery, no system has been provided for wide-spread surgical use which would allow such procedures to be performed safely within the prescribed time limits.

Recent advances in medical imagining technology coupled with advances in computer-based image processing and modeling capabilities have given physicians an unprecedented ability to visualize anatomical structures in live patients, and to use this information in diagnosis and treatment planning. The precision of image-based pre-surgical planning often greatly exceeds the precision of actual surgical execution. Precise surgical execution has been limited to procedures, such as brain biopsies, in which a suitable stereotactic frame is available. The inconvenience and restricted applicability of such a frame or device has led many researchers to explore the use of robotic devices to augment a surgeon's ability to perform geometrically precise tasks planned from computed tomography (CT) or other image data. The ultimate goal of the research is a partnership between man (the surgeon) and machines (computers and robots) that seeks to exploit the capabilities of both in order to better perform the task than can be accomplished alone by either man or machine.

Machines are very precise and untiring and can be equipped with any number of sensory feedback devices. Numerically controlled robots can move a surgical instrument through an exactly defined trajectory with precisely controlled forces. On the other hand, surgeons are very dexterous. They are also quite strong, fast, and are highly trained to exploit a variety of tactile, visual, and other cues. “Judgmentally” controlled, a surgeon understands surgical techniques and uses dexterity, senses, and experience to execute the procedure. However, the surgeon usually wants to be in control of everything that goes on. If the surgeon is interested in increasing his precision within acceptable limits of time or with sufficient speed, the surgeon must be willing to rely on machines to provide the precision.

Such less invasive attempts for positioning bypass grafts at target vessel locations have used small ports to access the anatomy. These approaches use endoscopic visualization and modified surgical instruments (e.g. clamps, scissors, scalpels, etc.) to position and suture the ends of the bypass graft at the host vessel locations. Attempts to eliminate the need for cardiopulmonary bypass support while performing CABG procedures have benefited from devices that stabilize the motion of the heart, retractors that temporarily occlude blood flow through the host vessel, and shunts that re-route the blood flow around the anastomosis site. Stabilizers and retractors still require significant time and complexity to expose the host vessel and suture the bypass graft to the host vessel wall. Shunts not only add to the complexity and length of the procedure, but they require a secondary procedure to close the insertion sites proximal and distal to the anastomosis site.

Attempts to automate the formation of sutureless anastomoses have culminated into mechanical stapling devices. Mechanical stapling devices have been disclosed for creating end-end anastomoses between the open ends of transected vessels. The Berggren et al. patents disclose an automatic stapling device for use in microsurgery (see, e.g., U.S. Pat. Nos. 4,607,637, 4,624,257, 4,917,090, and 4,917,091). The stapling device includes mating sections containing pins that are locked together after the vessel ends are fed through lumens in the sections and everted over the pins. The stapling device maintains intima-to-intima apposition for the severed vessel ends but has a large profile and requires impaling the everted vessel wall with the pins.

U.S. Pat. No. 4,214,587 to Sakura describes a mechanical end-end stapling device designed to reattach severed vessels. The device has a wire wound into a zigzag pattern to permit radial motion and contains pins bonded to the wire that are used to penetrate tissue. One vessel end is everted over and secured to the pins of the end-end stapling device, and the other vessel end is advanced over the end-end stapling device and attached with the pins.

Another mechanical end-end device that inserts mating pieces into each open end of a severed vessel is disclosed in U.S. Pat. No. 5,503,635 to Sauer et al. Once positioned, the mating pieces snap together to bond the vessel ends. The end-end devices are amenable to reattaching severed vessels but are not suitable to producing end-end anastomoses between a bypass graft and an intact vessel, especially when exposure to the vessel is limited.

Mechanical stapling devices have also been disclosed for end-side anastomoses. The devices are generally designed to insert bypass grafts, which can be attached to the mechanical devices, into the host vessel through a large incision and secure the bypass graft to the host vessel. The Kaster patents describe vascular stapling apparatus for producing end-side anastomoses. (See U.S. Pat. Nos. 4,366,819, 4,368,736, and 5,234,447.) The end-side apparatus is inserted through a large incision in the host vessel wall. The apparatus has an inner flange that is placed against the interior of the vessel wall, and a locking ring that is affixed to the fitting and contains spikes that penetrate into the vessel thereby securing the apparatus to the vessel wall. The bypass graft is itself secured to the apparatus in the everted or non-everted position through the use of spikes incorporated in the apparatus design.

U.S. Surgical has developed automatic clip appliers that replace suture stitches with clips (see, e.g., U.S. Pat. Nos. 5,868,761, 5,868,759, and 5,779,718). The clipping devices have been demonstrated to reduce the time required to produce the anastomosis but still require creating a large incision through the host vessel wall. As a result, blood flow through the host vessel must be interrupted while creating the anastomosis.

U.S. Pat. No. 5,695,504 to Gifford et al. discloses an end-side stapling device that secures harvested vessels to host vessel walls while maintaining intima-to-intima apposition. The stapling device is also inserted through a large incision in the host vessel wall and uses staples incorporated in the device to penetrate into tissue and secure the bypass graft to the host vessel.

The Walsh et al. patents disclose a similar end-side stapling device. (See U.S. Pat. Nos. 4,657,019, 4,787,386, and 4,917,087.) The end-side device has a ring with tissue piercing pins. The bypass graft is everted over the ring; the pins then penetrate the bypass graft thereby securing the bypass graft to the ring. The ring is inserted through a large incision created in the host vessel wall and the tissue piercing pins are used to puncture the host vessel wall. A clip is then used to prevent dislodgment of the ring relative to the host vessel.

End-side stapling devices require insertion through a large incision, which dictates that blood flow through the host vessel must be interrupted during the process. Even though these and other clipping and stapling end-side anastomotic devices have been designed to decrease the time required to create the anastomosis, interruption of blood flow through the host vessel increases the morbidity and mortality of bypass grafting procedures, especially during beating heart CABG procedures. A recent experimental study of the U.S. Surgical ONE-SHOT anastomotic clip applier observed abrupt ventricular fibrillation during four of fourteen internal thoracic artery to left anterior descending artery anastomoses in part due to coronary occlusion times exceeding 90 seconds (Heijmen et al: “A Novel One-Shot Anastomotic Stapler Prototype for Coronary Bypass Grafting on the Beating Heart: Feasibility in the Pig” J Thorac Cardiovasc Surg. 117:117-25; 1999). It would therefore be useful to develop a device for inserting a suitable patch into cardiac or other tissue that overcomes the above problems.

### SUMMARY OF THE INVENTION

According to the present invention, there is provided a deployment device for deploying a material into a patient, said deployment device including a housing and a placement device. The placement device includes a retracted condition within the housing for holding a material in a collapsed condition within the housing and an extended condition from the housing for disposing and releasing the material at a predetermined site in an uncollapsed condition. A method of deploying a material includes the steps of actuating the placement device to the extended condition and affixing the material to the extended placement device, retracting the placement device into the housing with the material in a collapsed condition, extending the placement device, and placing the material at a predetermined site in an uncollapsed condition.

### BRIEF DESCRIPTION OF THE DRAWINGS

Other advantages of the present invention are readily appreciated as the same becomes better understood by reference to the following detailed description when considered in connection with the accompanying drawings wherein:

FIGS. 1A and B are side views of one embodiment of a deployment device of the present invention;

FIG. 2 is a side view partially cut away of the deployment device of the present invention contained within a housing;

FIGS. 3A and B are side views showing a tube system for affixing material to an alternative embodiment of a deployment device, FIG. 3B is an enlarged view of the tube system shown in FIG. 3A;

FIGS. 4A and B are side views showing a band system for affixing material to the deployment device, FIG. 4B is an enlarged view of the band system shown in FIG. 4A;

FIGS. 5A and B are side views showing a wire system for affixing material to the deployment device, FIG. 5B is an enlarged view of the wire system shown in FIG. 5A;

FIGS. 6 A through D are enlarged views of the attachment devices used to attached material to the deployment device;

FIGS. 7A through C are side and enlarged views of the wire system for affixing material to the deployment device, and sutures from the ring that can be used to attach the patch to the ring deployment system with the same release mechanism;

FIGS. 8A and B are enlarged side views of the wire system for affixing material to the deployment device;

FIG. 9 is a side view of material affixed to the deployment device of the present invention;

FIG. 10 is a top view of material affixed to the deployment device of the present invention;

FIG. 11 is a plan view of the deployment device as a ring with a suture for affixing implantable material to the ring;

FIG. 12 is a plan view of the deployment device as a ring with a suture for affixing implantable material to the ring, where the suture is partially removed;

FIG. 13 is a plan view of the deployment device as a ring with a suture for affixing implantable material to the ring, where the suture is partially removed; and

FIG. 14 is a side view of the deployment device wherein the device is shaped as a spatula.

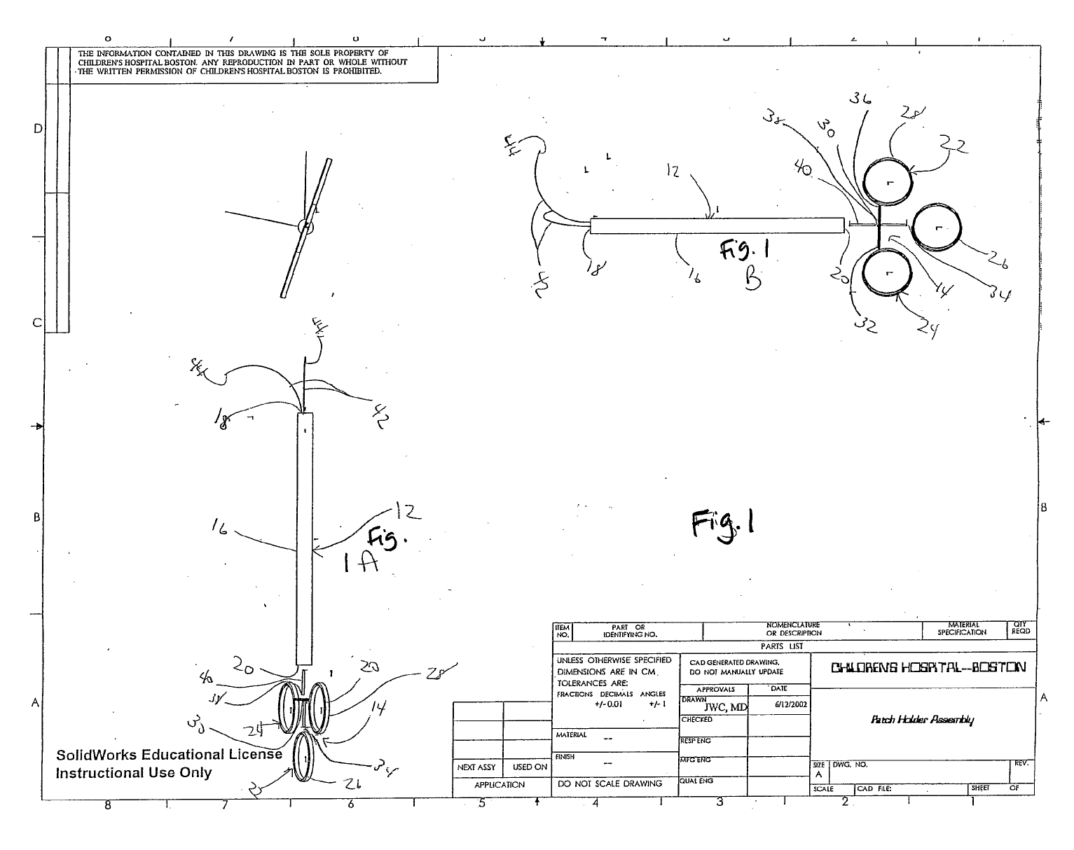

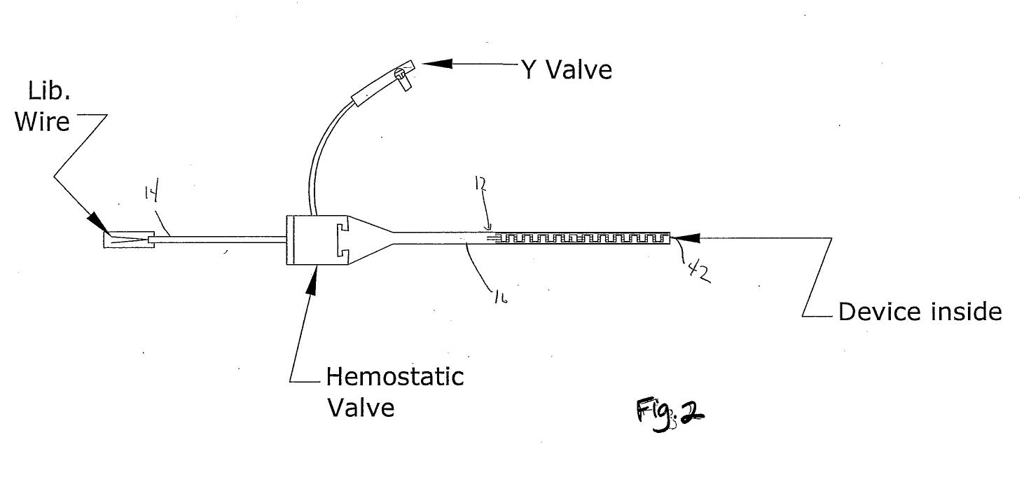

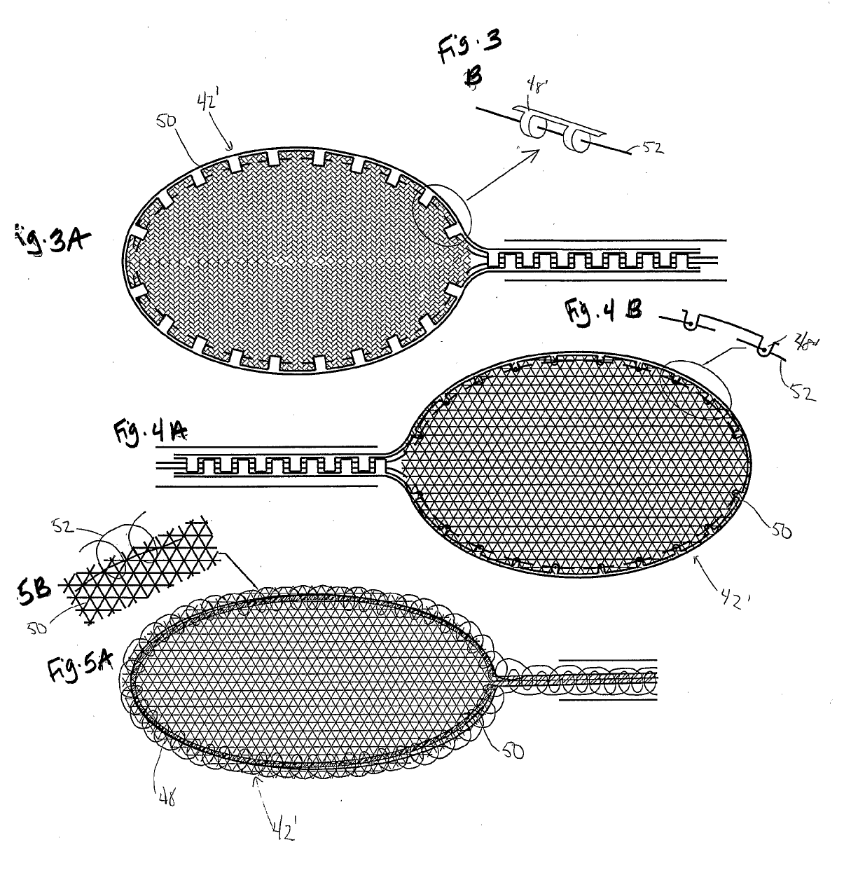

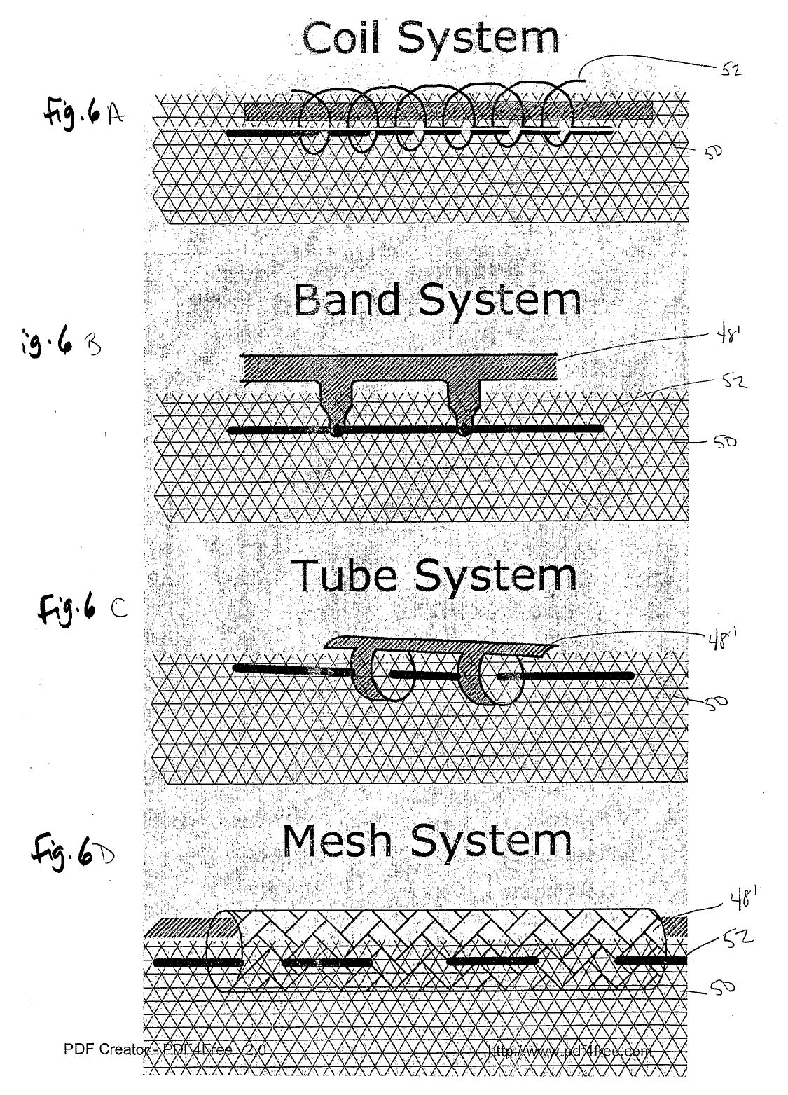

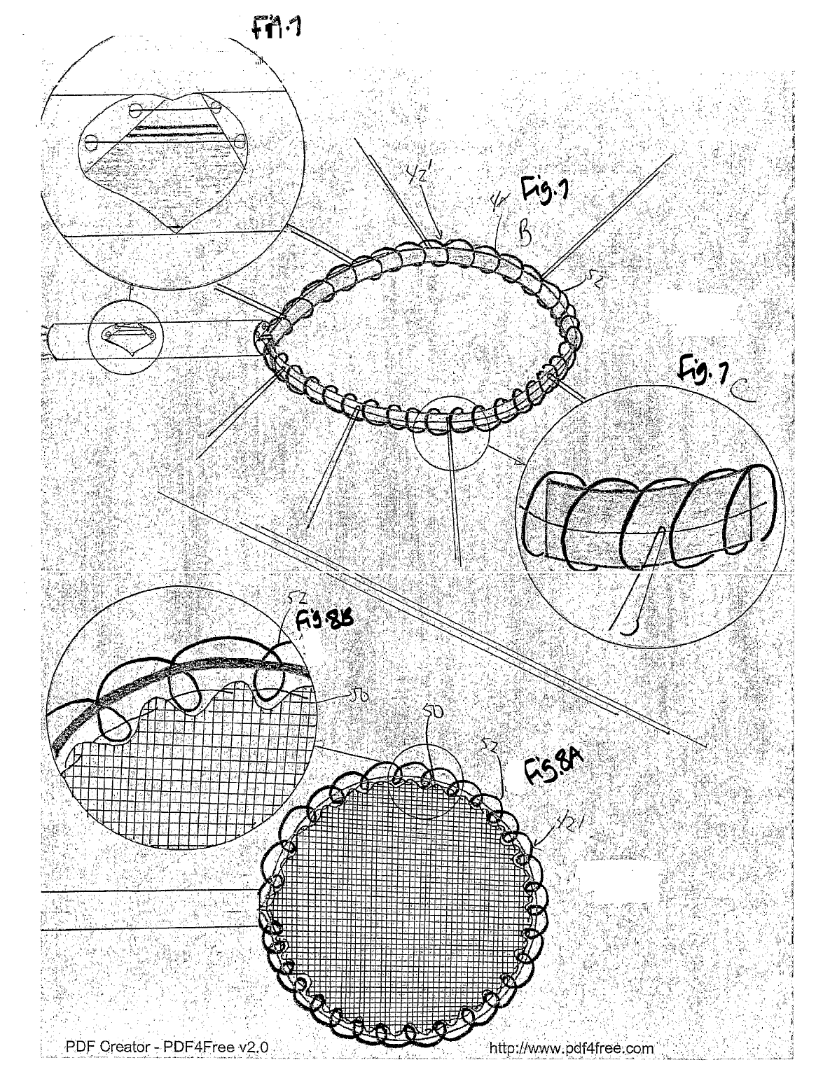

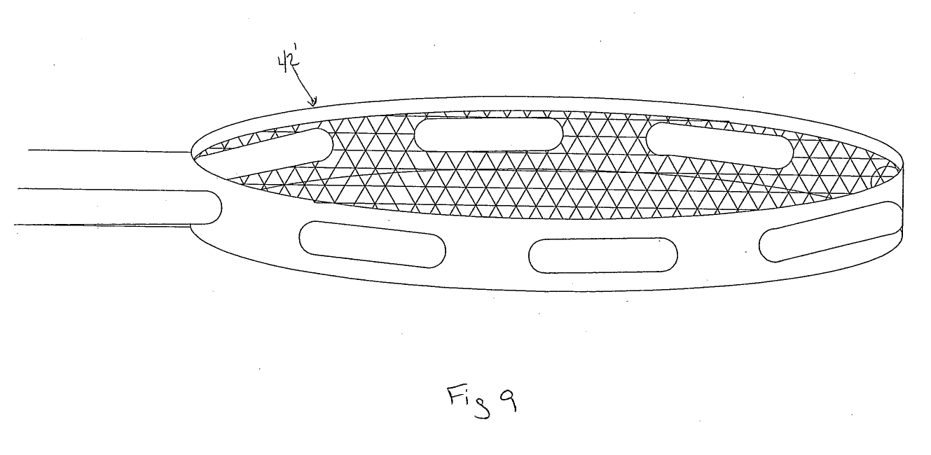

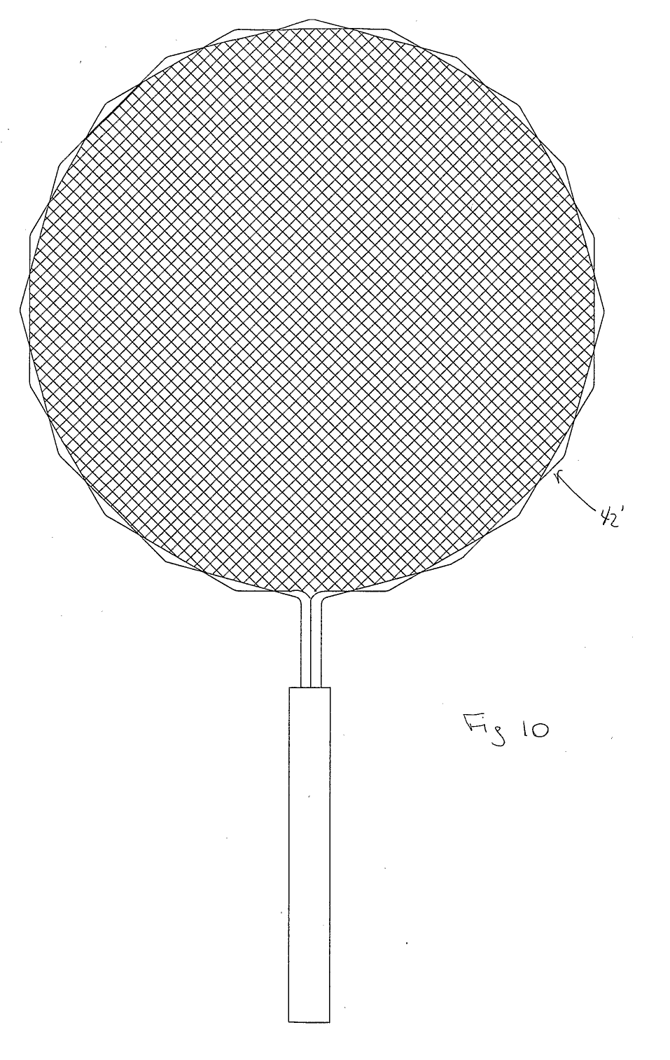

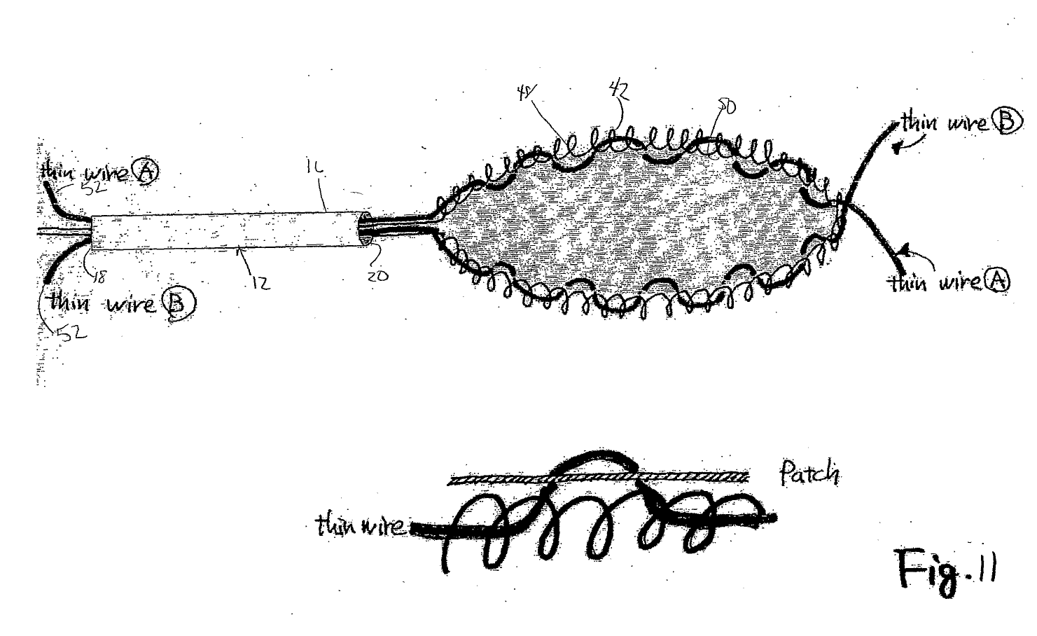

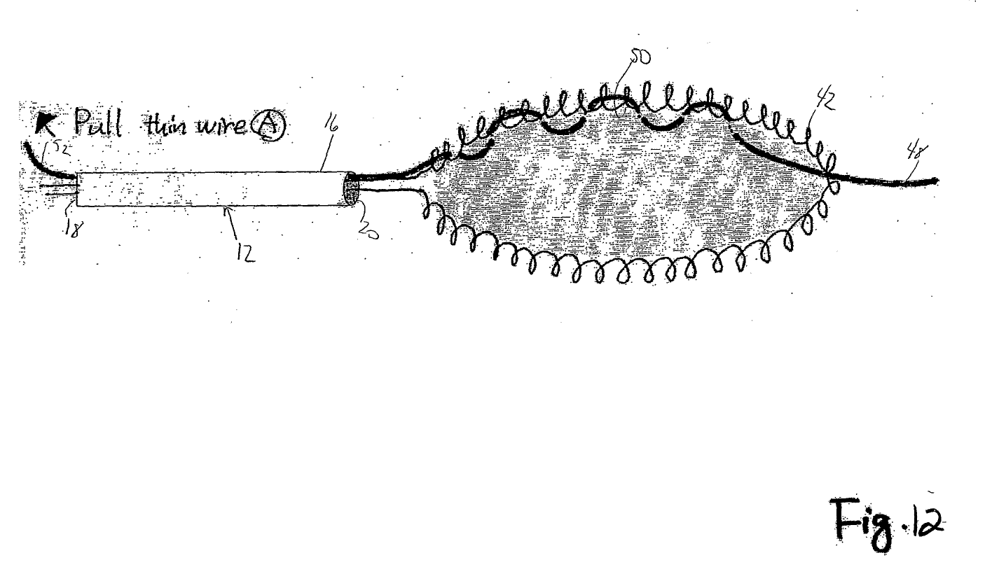

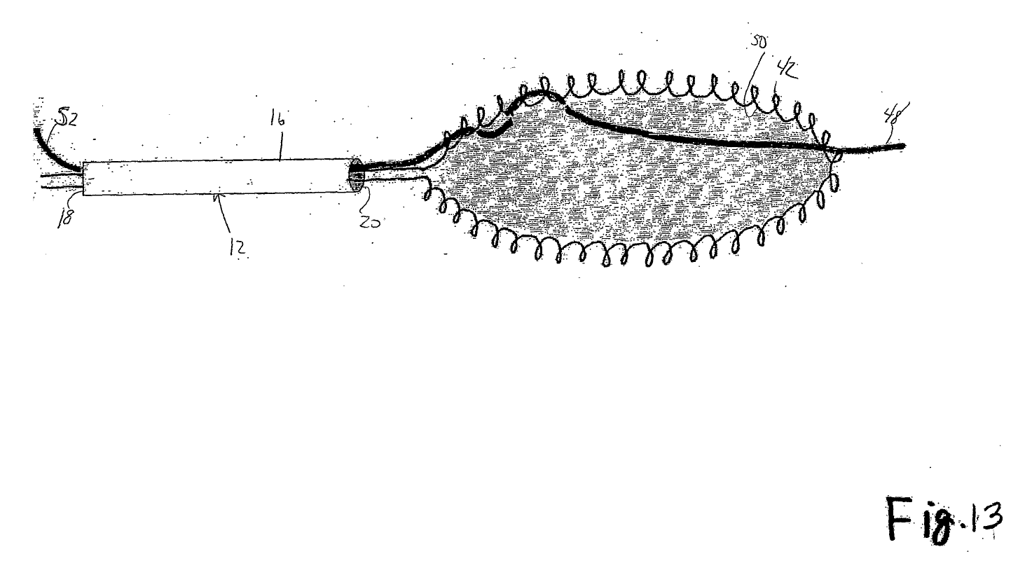

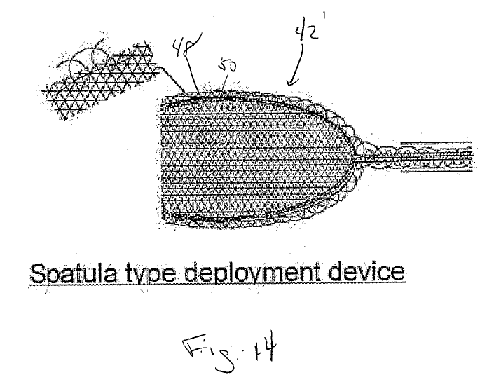

### DESCRIPTION OF THE INVENTION

Generally, the present invention provides a deployment device for deploying bioprosthetic or synthetic materials or analogous body tissue into a body of a patient.

More specifically, the deployment device 10 of the present invention includes a housing or cannula 12 and an insertion device 14. The housing 12 and insertion device 14 are connected such that the insertion device 14 is disposed within a lumen 16 of the housing 12. Preferably, the housing 12 and insertion device 14 are formed of two separate pieces of material.

The lumen 16 of the housing 12 includes two ends, an insertion end 16 for inserting into the body of the patient and an opposite end 18 in which the insertion device 14 is disposed. The housing 12 of the present invention is preferably formed in a manner known to those of skill in the art using a resilient material, such as 304 or 316 stainless steel. While steel is the preferred material, any resilient material that can be formed containing the structures disclosed herein can be used.

The interior of the lumen 16 is hollow thus enabling the insertion device 14 to be disposed within the lumen 16. The lumen 16 is large enough to contain a bioprosthesis or patch. Further, the lumen 16 can be in any shape that is capable of holding therein the material. For example, the lumen 16 can be cylindrical, square, rectangular, oval, or triangular.

The insertion device 14 of the present invention includes a handle 22 for controlling the insertion device 14. The handle 22 can be in any form that is capable of being attached to the insertion device 14 of the present invention as long as the handle 22 provides the surgeon with the ability to retract or extend the insertion device 14 and material being held by the insertion device 14. For example, the handle 22 can includes at least two, and preferably three loops 24, 26, 28 that are sized to allow the insertion of fingers therein. The loops 24, 26, 28 are sized to enable the surgeon to insert two or three fingers into the loops 24, 26, 28. The loops 24, 26, 28 are preferably made of a resilient material that is not malleable and therefore cannot be easily bent during use. Examples of such materials include, but are not limited to, hard plastics and solid metals, such as steel.

The loops 24, 26, 28 are arranged, as shown in the figures, via a t-bar 30, which is a t-shaped portion of the device. The t-bar 30 is configured such that one of the loops 24, 26, 28 is located at three 32, 34, 36 of the four ends of the t-bar 30. The t-bar 30 is formed of a resilient, non-malleable material. Preferably, the t-bar 30 is formed of the same material that is used in making the loops 24, 26, 28. On the fourth end 38 of the t-bar 30 there is located a retractable rod 40.

The rod 40 is disposed within the lumen 16 of the housing 12 and extends from the fourth end 38 of the t-bar 30 to the insertion end 20 of the housing 12. The rod 40 is formed of a resilient material that does not bend easily. Examples of such materials are well known to those of skill in the art. The end of the rod 40 that exits the insertion end 20 includes multiple material holding devices 42. The holding devices 42 are formed of resilient, but pliable material, such that the holding devices 42 can extend outward in a curved umbrella shape, as shown in the figures, or can be held in a straight position, i.e. when the rod 40 is retracted into the lumen 16 of the housing 12. There are preferably six to eight holding devices 42.

The holding devices 42 are shown in the form of curvate, radially outwardly extending spokes when in the extended condition. They easily conform to the inner shape of the lumen 16 when retracted therein.

On the ends of the holding devices 42 are spires 44. Spires are devices that are able to hold and subsequently release the material to be inserted into the body in place without damaging the material. Preferably, the spires are formed as a barb.

Alternatively, the holding device 42 can be formed as a ring 42′. The ring 42′ can be formed of a resilient self-expanding, self-contracting material, such materials are well known to those of skill in the art. The ring 42′ is preferably formed of a memory-type material or spring-like material that conforms to the shape of the lumen but can expand when extended outside of the lumen 16. The ring 42′ can be formed of any resilient self-expanding, self-contracting material, including, but not limited to, nitinol, elgiloy, and other shape-memory metals. The shape memory metal can be formed of any suitable, biocompatible shape memory metal known to those of skill in the art. Examples of shape memory metals that can be used include, but are not limited to, nickel-titanium alloy, generically known as nitinol, elgiloy, copper-aluminum-nickel, copper-zinc-aluminum and iron-manganese-silicon alloys. Preferably, the shape memory metal material is made of nitinol. Nitinol has two phases, a martensitic phase and an austenitic phase. A ring 42′ of nitinol can be formed to a desired shape such as that shown in FIG. 3. The shape is heat set into position. The nitinol is then cooled while maintaining its shape. The shape can be plastically deformed to a new shape. Upon subsequent heating, the metal returns to the original shape. There are no limitations on the size, diameter, thickness, or shape of the ring 42′.

The shape memory material is secured to the rod 40 by crimping a portion 46 of a shape memory ring 42′ over the end of the rod 40 that exits the insertion end 20. The ring 42′ is then secured, such as by gluing. Alternatively, the shape memory ring 42′ can be secured to the rod 40 using other methods known to those of skill in the art for affixing shape memory alloys to other materials.

The ring 42′ also includes a gripping device 48. The gripping device 48 can include any material capable of holding and maintaining the material on the ring 42′ without adversely altering the shape memory material of the ring 42′. One example of such a gripping device is a suture. The suture is constructed from a biocompatible material. The sutures can be monofilaments or multifilaments (e.g. braided). Suitable materials include, but are not limited to, polypropylene, Dacron™, polyester, Gortex™, nylon, 7-0 prolene, 8-0 prolene, and 4-0 nylon. Commercial examples include Ethibond Excel™ polyester fiber sutures, Ethilon™ nylon sutures, Mersilene™ polyester fiber sutures, Nurolon nylon sutures, and Prolene polypropylene sutures, each available from Ethicon. The material of the gripping device 48 can be bioresorbable or non-bioresorbable (e.g. substantially permanent). As used herein, absorbable filament means a sterile strand prepared from a substance (e.g. collagen) derived healthy mammals or a synthetic polymer. Bioresorbable filaments can be constructed from materials of biological origin (e.g. surgical gut) and are digestable by tissue enzymes. Alternatively, a bioabsorbable filament can be constructed from a synthetic polymer that can be broken down by hydrolysis or a shape memory polymer. The absorbable filament can be treated or constructed to modify its resistance to absorption. The filament that forms the gripping device 48 can also include an antimicrobial agent. The gripping device 48 can also be formed as a band, tube, or piece of mesh as shown in FIGS. 3 through 6.

Alternatively, the holding device 42 can be formed as a spatula 42″. The spatula 42″ can be formed of a resilient self-expanding, self-contracting material, such materials are well known to those of skill in the art. The spatula 42″ is preferably formed of a memory-type material or spring-like material that conforms to the shape of the lumen but can expand when extended outside of the lumen 16. The spatula 42″ can be formed of any resilient self-expanding, self-contracting material, including, but not limited to, nitinol, elgiloy, and other shape-memory metals. The shape memory metal can be formed of any suitable, biocompatible shape memory metal known to those of skill in the art. Examples of shape memory metals that can be used include, but are not limited to, nickel-titanium alloy, generically known as nitinol, elgiloy, copper-aluminum-nickel, copper-zinc-aluminum and iron-manganese-silicon alloys. Preferably, the shape memory metal material is made of nitinol. Nitinol has two phases, a martensitic phase and an austenitic phase. A spatula 42″ of nitinol can be formed to a desired shape such as that shown in FIG. 14. The shape is heat set into position. The nitinol is then cooled while maintaining its shape. The shape can be plastically deformed to a new shape. Upon subsequent heating, the metal returns to the original shape. There are no limitations on the size, diameter, thickness, or shape of the spatula 42″.

The shape memory material is secured to the rod 40 by crimping a portion 46 of a shape memory spatula 42″ over the end of the rod 40 that exits the insertion end 20. The spatula 42″ is then secured, such as by gluing. Alternatively, the shape memory spatula 42″ can be secured to the rod 40 using other methods known to those of skill in the art for affixing shape memory alloys to other materials.

The spatula 42″ also includes a gripping device 48. The gripping device 48 can include any material capable of holding and maintaining the material on the spatula 42″ without adversely altering the shape memory material of the spatula 42″. One example of such a gripping device is a suture. The suture is constructed from a biocompatible material. The sutures can be monofilaments or multifilaments (e.g. braided). Suitable materials include, but are not limited to, polypropylene, Dacron™, polyester, Gortex™, nylon, 7-0 prolene, 8-0 prolene, and 4-0 nylon. Commercial examples include Ethibond Excel™ polyester fiber sutures, Ethilon™ nylon sutures, Mersilene™ polyester fiber sutures, Nurolon nylon sutures, and Prolene polypropylene sutures, each available from Ethicon. The material of the gripping device 48 can be bioresorbable or non-bioresorbable (e.g. substantially permanent). As used herein, absorbable filament means a sterile strand prepared from a substance (e.g. collagen) derived healthy mammals or a synthetic polymer. Bioresorbable filaments can be constructed from materials of biological origin (e.g. surgical gut) and are digestable by tissue enzymes. Alternatively, a bioabsorbable filament can be constructed from a synthetic polymer that can be broken down by hydrolysis or a shape memory polymer. The absorbable filament can be treated or constructed to modify its resistance to absorption. The filament that forms the gripping device 48 can also include an antimicrobial agent.

The deployment device 10 of the present invention can be used with a trocar for the introduction of a bioprosthetic or synthetic material, such as a patch. Examples of bioprosthetics include, but are not limited to, autologous pericardium, a collapsed valve, a baffle, or other prosthetic reinforcement.

The deploying device 10 functions as follows. The insertion device 22 is actuated to the extended condition. When the insertion device is extended, the holding devices 42 extend radially outwardly and away from each other. The shape of the radial extension depends upon the requirements of the materials being affixed thereto. A material to be placed within the body of a patient is placed on the spires 44 of the holding devices 42 of the insertion device 22. The rod 40 is then retracted into the lumen 16 of the housing 12, thereby collapsing the material within the housing 12. The deployment device 10 can then either be inserted into a trocar, inserted directly into a body, or placed at a predetermined site that requires the attached material. Once the deployment device 10 is inserted into the trocar, or other location, the insertion device 14 is depressed, thereby extending the rod 40 outside of the trocar and into the body of the patient. When the material is placed in the proper location, then the material can be released by the spires 44 and affixed in the proper location in an uncollapsed condition.

Alternatively, when a ring 42′ or spatula 42″ is used as the holding device 42, the device 10 functions as follows. The insertion device 22 is actuated to the extended condition. When the insertion device is extended, the ring 42′ or spatula 42″ expands. The shape of the expansion depends upon the requirements of the materials being affixed thereto. A material to be placed within the body of a patient is placed on the gripping device 48 of the ring 42′ or spatula 42″ of the insertion device 22. When the gripping device 48 is a suture, the suture is sewn through the perimeter 50 of the material. Such affixing can occur either by hand or automatically. The ring 42′ or spatula 42″ is then retracted enabling the rod 40 to be retracted into the lumen 16 of the housing 12, thereby collapsing the material within the housing 12. The deployment device 10 can then either be inserted into a trocar, inserted directly into a body, or placed at a predetermined site that requires the attached material. Once the deployment device 10 is inserted into the trocar, or other location, the insertion device 14 is depressed, thereby extending the rod 40 outside of the trocar and into the body of the patient. When the material is placed in the proper location, then the material can be released by the gripping device 48 and affixed in the proper location in an uncollapsed condition. When the gripping device 48 is a suture, the ends of the suture 52, which extend through the lumen 16 of the housing 12 are pulled thereby withdrawing the suture from the material. Preferably, prior to removing the suture, staples or other fixing devices are used to affix the material in place within the patient.

For example, autologous pericardium or a 0.1 mm polytetrafluoroethylene (PTFE) patch (Gore-Tex, W. L. Gore & Associates, Inc, Flagstaff, Ariz.) can be trimmed and then sutured along the ring 42′ with 8-0 prolene (Ethicon Inc., Somerville, N.J.). The device 10 can be delivered through a trocar (5 mm in diameter), and the ring 42′, with autologous pericardium, can be extended out of the trocar and allowed to expand.

An example of when the present invention can be used is during closed heart cardiac surgery. During closed heart cardiac surgery there are times when a patch is needed in a vessel or on the inner wall of the chamber of the heart. The present invention can be used to dispose such a patch, in situ, without need of by-pass open-heart surgery. The patch can be delivered, disposed, and released using the present invention.

Throughout this application, author and year, and patents, by number, reference various publications, including United States patents. Full citations for the publications are listed below. The disclosures of these publications and patents, in their entireties are hereby incorporated by reference into this application in order to more fully describe the state of the art to which this invention pertains.

The invention has been described in an illustrative manner, and it is to be understood that the terminology that has been used is intended to be in the nature of words of description rather than of limitation.

Obviously, many, modifications and variations of the present invention are possible in light of the above teachings. It is, therefore, to be understood that within the scope of the described invention, the invention may be practiced otherwise than as specifically described.

What is claimed is:

1. A deployment device for deploying a material into a patient, said deployment device comprising:
a housing; and
placement means having a retracted condition within said housing for holding a material, in a collapsed condition, within said housing and an extended condition from said housing for disposing and releasing the material at a predetermined site in an uncollapsed condition.

2. The device according to claim 1, wherein said housing includes an insertion end and an opposite end.

3. The device according to claim 2, wherein said housing includes a lumen connecting said insertion end and said opposite end.

4. The device according to claim 3, wherein said placement means is disposed in said insertion end of said housing and through said lumen.

5. The device according to claim 1, wherein said placement means includes controlling means for controlling the movement of said device.

6. The device according to claim 5, wherein said controlling means are finger loops.

7. The device according to claim 1, wherein said placement means includes holding means for holding the material on the placement means.

8. The device according to claim 7, wherein said holding means include curvate, radially, outwardly extending arms.

9. The device according to claim 8, wherein said holding means comprises an umbrella shaped wire.

10. The device according to claim 7, wherein said holding means further includes spires attached to ends of said holding means, said spires holding the material in place.

11. The device according to claim 1, wherein said placement means includes a self-expanding ring.

12. The device according to claim 11, wherein said ring is formed as a coiled loop.

13. The device according to claim 11, wherein said ring includes gripping means for maintaining the material on said ring.

14. The device according to claim 13, wherein said gripping means are sutures.

15. The device according to claim 14, wherein said sutures are formed of a material selected from the group consisting essentially of 8-0 prolene, 7-0 prolene, 4-0 nylon sutures.

16. The device according to claim 13, wherein said gripping means is at least one wire.

17. The device according to claim 16, wherein said wire further includes a needle for threading said wire through the material.

18. The device according to claim 17, wherein said wire is a thin flexible wire.

19. The device according to claim 18, wherein said wire is formed of a shape memory alloy.

20. The device according to claim 11, wherein said ring is formed of a shape memory alloy.

21. The device according to claim 20, wherein said shape memory alloy is selected from the group consisting essentially of nitinol and elgiloy, copper-aluminum-nickel, copper-zinc-aluminum and iron-manganese-silicon alloys.

22. The device according to claim 1, wherein said device is sized to fit within a trocar.

23. A method of deploying a material into a patient by:
actuating the placement means of claim 1 to an extended condition and affixing a material to the extended placement device;
retracting the placement device into the housing with the material in a collapsed condition;
inserting the deploying device into the body of a patient;
extending the placement device into the extended condition in the body; and
placing the material at a predetermined site in the uncollapsed condition.

24. The method according to claim 23, wherein said inserting step includes inserting the deployment device into a trocar placed within the body.

25. The method according to claim 24, wherein said affixing step includes threading a wire through the material to affix the material to the extended placement device.

26. The method according to claim 25, further including the step of removing the wire from the material after the material is placed at the predetermined location.

27. A method of deploying a material into a patient by:
inserting the material, in a collapsed condition, in a cavity of a patient through an opening made into the cavity;
expanding the material into an uncollapsed condition; and placing the material at a predetermined site.

28. A method as set forth in claim 27, further including the step of covering and patching over an opening in the cavity with the uncollapsed material.
